(19) United States
(12) Patent Application Publication
(10) Pub. No.: US 2024/0138666 A1
(43) Pub. Date: May 2, 2024

(54) INSERTION DEVICE WITH DISTAL CHAMBER

(71) Applicant: Boston Scientific Scimed, Inc., Maple Grove, MN (US)

(72) Inventor: Michael S.H. CHU, Brookline, MA (US)

(73) Assignee: Boston Scientific Scimed, Inc., Maple Grove, MN (US)

(21) Appl. No.: 18/493,283

(22) Filed: Oct. 24, 2023

Related U.S. Application Data
(60) Provisional application No. 63/381,233, filed on Oct. 27, 2022.

(51) Int. Cl. A61B 1/307; A61B 18/24; A61B 18/26

(52) U.S. Cl. CPC A61B 1/307; A61B 18/245; A61B 18/26; A61B 2018/00172

(57) ABSTRACT

A system includes an adapter and an insertion device. The device includes a shaft extending longitudinally from a proximal end to a distal end. The shaft includes first and second working channels extending therethrough. The adapter includes a proximal portion coupled to the distal end and a distal portion defining a chamber receiving a stone. The proximal portion includes an outlet extending through a portion thereof positioned so that, when the adapter is in desired alignment, a fluid supplied to the first channel is directed out of the adapter via the outlet to an area surrounding the adapter. The adapter further includes an inlet channel extending longitudinally through the proximal portion to a distal opening which is open to the chamber. The inlet channel is configured so that, when negative pressure is applied thereto, the fluid is drawn through the distal opening through the inlet channel to the second channel.

### CROSS-REFERENCE TO RELATED APPLICATIONS

This application claims the benefit of priority to U.S. Provisional Application No. 63/381,233, filed on Oct. 27, 2022, which is incorporated by reference herein in its entirety.

### BACKGROUND

Insertion devices, including scope devices, may be used in conjunction with energy devices and/or retrieval devices to retrieve foreign bodies or bodily debris such as stones, stone fragments, or tissue from within the human body. For example, ureteroscopes, such as the LithoVue™ flexible ureteroscope, are often used in the treatment of kidney and ureteral stones. Ureteroscopes are typically used in conjunction with devices such as guidewires, retrieval devices, and laser fibers. As an example, urologists will often use a ureteroscope in combination with a laser fiber used to pulverize kidney stones and a basket to retrieve and/or remove the debris from the body.

According to another example in which a dusting technique is utilized, urologists may use low-pulse, high-frequency laser energies of more powerful laser systems to pulverize stones into extremely fine fragments so that the fragments may be left behind in the kidney (to pass spontaneously in the urine) or removed from the body via suction. Stone dust in the lower poles, however, will most likely not drain or pass spontaneously. Thus, urologists will relocate the lower pole stones to the upper poles prior to dusting.

Techniques utilizing insertion devices such as ureteroscopes, however, may be time consuming and may require multiple scope manipulations/deflections in order to fragment and capture stones for removal. For example, stone fragmentation may require additional time and effort to locate, capture and remove the fragments since stone fragments less than 3 mm may be difficult to basket, capture, and remove. In addition, the basket and scope must be removed from the body for each stone retrieval. In some cases, a large stone that is misjudged in size may become wedged or stuck in the ureters or an access sheath. In other cases, fragmenting renal stones distal to the scope may scatter dust throughout the kidney. In addition, lasering often has a retropulsion effect in which the laser energy pushes the stone away from the laser, thereby requiring the laser to be repositioned often so that the laser may be in contact with the stone to have an optimum fragmenting effect. Rapid lasering, however, may result in overheating of the adjacent fluids to burn/scorch surrounding tissue.

### SUMMARY

The present disclosure relates to a system for treating stones in a hollow organ or body passage. The system includes an insertion device including a shaft extending longitudinally from a proximal end to a distal end. The shaft is configured for insertion to a target area within a hollow organ or a body passage and including a first working channel and a second working channel extending therethrough.

The system also includes an adapter including a proximal portion configured to be coupled to the distal end of the shaft in a desired alignment and a distal portion defining a chamber therein for receiving a target stone to be treated. The proximal portion includes an outlet extending through a portion thereof positioned so that, when the adapter is in the desired alignment, fluid supplied to the first working channel is directed out of the adapter via the outlet to an area surrounding the adapter. The adapter further includes an inlet channel extending longitudinally through the proximal portion to a distal opening which is open to the chamber, the inlet channel being configured so that, when negative pressure is applied thereto, fluid from the area surrounding the adapter is drawn through the distal opening through the inlet channel to the second working channel.

In an embodiment, the adapter includes a substantially tubular body extending from a proximal end to a distal end, a partition extending across a lumen of the substantially tubular body to define the proximal and distal portions.

In an embodiment, the outlet includes an aperture extending through a wall along the proximal portion of the adapter.

In an embodiment, the adapter includes a device channel extending through the proximal portion to a distal opening that is open to the chamber, the device channel configured to receive an energy device therethrough for applying an energy to a target stone received within or against the chamber.

In an embodiment, the proximal portion of the adapter incudes an alignment feature configured to engage a corresponding portion of a mounting bracket attached to the distal end of the shaft so that the adapter is coupled to the shaft in an operative configuration.

In an embodiment, the distal portion of the adapter has a smaller cross-sectional area than the proximal portion, the outlet configured as an outlet channel extending longitudinally through the proximal portion to a distal opening extending through a distal face of the proximal portion which extends beyond a periphery of the distal portion.

In an embodiment, the proximal and distal portions of the adapter are configured to be releasably coupled to one another.

In an embodiment, a distal portion of the adapter is formed of a translucent material.

In an embodiment, the proximal portion of the adapter includes a window sized and shaped to receive an imaging system of the insertion device therein so that the chamber is within a field of view of the imaging system.

In an embodiment, the chamber includes a tapered surface tapering toward a proximal end of the chamber to funnel any stones received therein toward a central axis along which the outlet channel extends.

In an embodiment, the at least a distal portion of the outlet channel has a non-circular cross-sectional area configured to align an energy device received therein toward a central axis along which the inlet channel extends so that a negative force may be applied through the inlet channel, about the energy device.

In an embodiment, the non-circular cross-sectional area is one of an elliptical shape, an elongated slot, and a cross-shape.

In addition, the present disclosure relates to a system for treating stones in a hollow organ or body passage. The system includes a ureteroscope including a shaft extending longitudinally from a proximal end to a distal end. The shaft is configured to be inserted to a target area within a hollow organ or a body passage and including a first working channel and a second working channel extending therethrough.

The system also includes an adapter including a proximal portion configured to be coupled to the distal end of the shaft in a desired alignment and a distal portion defining a chamber therein for receiving a target stone to be treated. The proximal portion includes an outlet extending through a portion thereof positioned so that, when the adapter is in the desired alignment, fluid supplied to the first working channel is directed out of the adapter via the outlet to an area surrounding the adapter. The adapter further includes an inlet channel extending longitudinally through the proximal portion to a distal opening which is open to the chamber, the inlet channel being configured so that, when negative pressure is applied thereto, fluid from the area surrounding the adapter is drawn through the distal opening through the inlet channel to the second working channel.

In an embodiment, the system further includes a laser fiber configured to be inserted through a third working channel of the ureteroscope to laser stones received within or against the chamber, the third working channel extending through the proximal portion to a distal opening that is open to the chamber.

In an embodiment, the system further includes an imaging system configured to facilitate navigation of the distal end of the shaft to the target area.

In addition, the present disclosure relates to a method for treating ureteral or kidney stones. The method includes inserting a scope device, with an adapter coupled to a distal end of a shaft of the scope device, to a target area within a patient's body, the adapter including a proximal portion configured to be coupled to the distal end of the shaft and a distal portion defining a chamber therein, the proximal portion including an outlet in fluid communication with a first working channel of the scope device and an inlet channel extending longitudinally through the proximal portion to a distal opening which is open to the chamber; applying a negative pressure through a second working channel so that a target stone is suctioned into or against a portion of the chamber; applying a laser energy to the target stone; and supplying a fluid to the target area via the outlet to provide a continuous fluid circulation from the outlet, proximally through the chamber and the second working channel to remove any stone particles and heat resulting from a lasering of the target stone from the chamber.

In an embodiment, the laser energy is provided via a laser fiber inserted distally through the shaft to the chamber via one of a device channel extending through the proximal portion of the adapter in communication with the chamber and the inlet channel of the adapter.

In an embodiment, the method further includes relocating the target stone, which is suctioned within or against the chamber, to a desired location within the patient's body for lasering.

In an embodiment, the supplying a fluid to the target area to provide the continuous fluid circulation includes managing the fluid circulation to control a pressure within the target area resulting from one of flushing and relocating one of the target stone and stone particles.

In an embodiment, the method further includes capturing one of stones, stone fragments, and particles too large to pass proximally through an outlet channel within the chamber for removal from the patient's body.

### BRIEF DESCRIPTION OF THE DRAWINGS

FIG. 1 shows a longitudinal side view of a distal portion of a system according to an exemplary embodiment of the present disclosure;

FIG. 2 shows a longitudinal side view of a scope device according to the system of FIG. 1;

FIG. 3 shows an enlarged view of a portion of a handle member of the scope device according to the system of FIG. 1;

FIG. 4 shows a longitudinal side view of the scope device of the system of FIG. 1, in a deflected configuration;

FIG. 5 shows a perspective view of an adapter according to the system of FIG. 1;

FIG. 6 shows a plan view of the adapter of FIG. 5;

FIG. 7 shows a perspective view of a distal portion of the system according to FIG. 1;

FIG. 8 shows a longitudinal side view of the distal portion of the system of FIG. 1;

FIG. 9 shows a perspective view of an adapter according to another exemplary embodiment of the present disclosure;

FIG. 10 shows a plan view of the adapter of FIG. 9;

FIG. 11 shows a cross-sectional view of a distal portion of the adapter of FIG. 9;

FIG. 12 shows a perspective view of an adapter according to yet another exemplary embodiment of the present disclosure, in an unassembled configuration;

FIG. 13 shows a longitudinal side view of the adapter according to FIG. 12, in the unassembled configuration;

FIG. 14 shows a longitudinal side view of the adapter according to FIG. 12, in an assembled configuration;

FIG. 15 shows a perspective view of a distal portion of a system according to another exemplary embodiment of the present disclosure;

FIG. 16 shows a plan view of an adapter according to the exemplary system of FIG. 15;

FIG. 17 shows a longitudinal side view of the distal portion of the system of FIG. 15;

FIG. 18 shows a cross-sectional view of an inlet channel of an adapter according to an alternate embodiment of the present disclosure;

FIG. 19 shows a cross-sectional view of an inlet channel of an adapter according to yet another alternate embodiment of the present disclosure;

FIG. 20 shows a perspective view of a distal portion of a system according to another exemplary embodiment of the present disclosure;

FIG. 21 shows a plan view of an adapter according to the exemplary system of FIG. 20;

FIG. 22 shows a perspective view of a distal portion of a system according to yet another exemplary embodiment of the present disclosure;

FIG. 23 shows a perspective view of a gateway adapter according to the exemplary system of FIG. 22;

FIG. 24 shows a longitudinal side view of a distal portion of a system according to another exemplary embodiment of the present disclosure;

FIG. 25 shows a longitudinal side view of a distal portion of a system according to yet another exemplary embodiment of the present disclosure;

FIG. 26 shows a perspective view of a distal portion of a system according to further exemplary embodiment of the present disclosure;

FIG. 27 shows a perspective view of a distal portion of a system according to another exemplary embodiment of the present disclosure, in an unassembled configuration;

FIG. 28 shows a perspective view of the distal portion of the system of FIG. 27, in an assembled configuration;

FIG. 29 shows a perspective view of a distal portion of a system according to another exemplary embodiment of the present disclosure, in an unassembled configuration;

FIG. 30 shows a perspective view of the distal portion of the system of FIG. 29, in an assembled configuration;

FIG. 31 shows a perspective view of a distal portion of a system according to yet another exemplary embodiment of the present disclosure, in an unassembled configuration;

FIG. 32 shows a perspective view of the distal portion of the system of FIG. 31, in an assembled configuration;

FIG. 33 shows a perspective view of a distal portion of a system according to another exemplary embodiment of the present disclosure, in an unassembled configuration; and

FIG. 34 shows a perspective view of the distal portion of the system of FIG. 33, in an assembled configuration.

### DETAILED DESCRIPTION

The present disclosure may be further understood with reference to the following description and appended drawings, wherein like elements are referred to with the same reference numerals. The present disclosure relates to systems and methods for accessing, examining and/or treating a hollow organ, a body passage, or cavity of a body and, in particular, may relate to systems and method for performing ureteroscopies and may, more particularly relate to the treatment of kidney stones.

The exemplary embodiments describe a system including a ureteroscope, which may be used for treating ureteral and/or kidney stones, and an adapter mountable to a distal end of a shaft the ureteroscope. The exemplary adapter is configured to provide an outlet, via which fluid may be provided to a target area, and an inlet path, via which a negative pressure may be applied, so that continuous fluid circulation is provided to transport fragments, particles, and/or debris along with any resulting heat and/or pressure out of the body and away from the target area.

Additionally, the adapter includes a distal chamber for receiving or otherwise confining a stone or stone fragment to be captured and/or lasered. It should be noted that although the exemplary embodiments show and describe systems and methods including ureteroscopes, it will be understood by those of skill in the art that the systems and methods of the present disclosure may include any of a variety of other insertion devices which may be similarly used in conjunction with other energy devices and/or retrieval devices and in other areas of the body. It should also be noted that the terms “proximal” and “distal,” as used herein, are intended to refer to a direction toward (proximal) and away from (distal) a user of the device (e.g., physician).

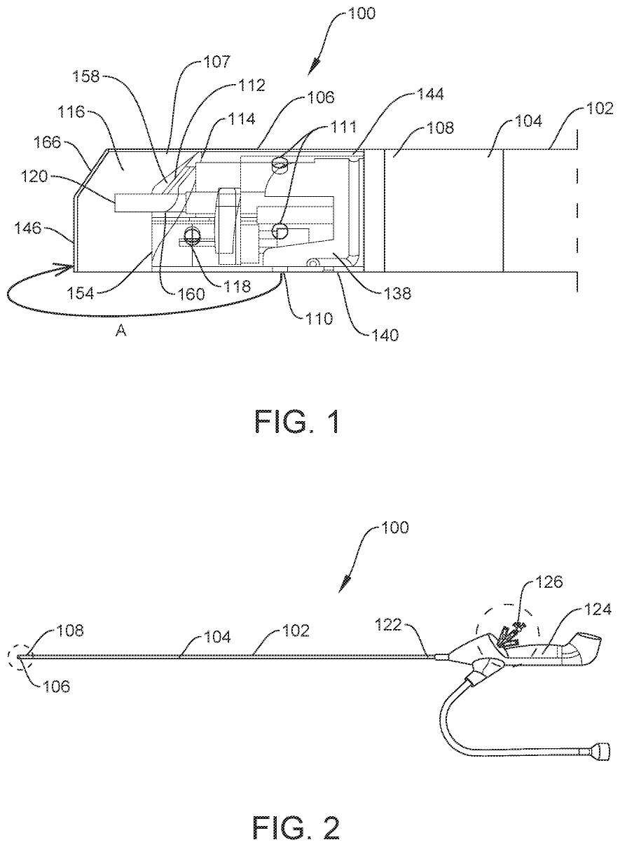

As shown in FIGS. 1-8, a system 100 for treating a hollow organ or cavity according to an exemplary embodiment of the present disclosure comprises a scope device 102 (e.g., ureteroscope) including a shaft 104 configured to be inserted through a body lumen to a target area (e.g., ureter, kidney accessed via a natural body orifice) and an adapter 106 permanently mounted on a distal end 108 of the shaft 104 of the scope device 102 so that each of a plurality of channels or openings extending through portions of the adapter 106 are in fluid communication with and/or aligned with corresponding portions of the scope device 102. The shaft 104 may be a flexible shaft.

In particular, the adapter 106 includes an outlet 110 via which fluid passed through the shaft 104 may be provided to the target area and an inlet channel 112 via which a negative pressure applied through the shaft 104 may be provided to the target area. The fluid provided through the outlet 110 and the negative pressure applied via the inlet channel 112 together provide a continuous fluid circulation along a path A, as shown in FIG. 1, through the target area so that stone fragments, particles and/or debris, along with any heated fluids (heated via the lasering of stones or other treatments), may be removed from the target area. The adapter 106 may thus also include a laser channel 114 through which a laser fiber 120 passed through a working channel of the shaft 104 may be received to laser a stone or stone fragment. The adapter 106 also includes a distal chamber 116 open to an exterior of the adapter 106 at a distal end 146 thereof for receiving a stone or stone fragment therein and/or thereagainst during removal, transport, and/or lasering of the stone. In an exemplary embodiment, a fluid management system of the scope device 102 may be used to control fluid supplied to and/or removed from the target area to manage, for example, an inter-renal pressure resulting from the flushing and/or transport of stones or stone fragments.

The scope device 102, as shown in FIGS. 1-4, may include any of a variety of scope devices including, for example, a ureteroscope, such as the LithoVue™ or LithoVue™ Elite. As shown in FIGS. 2 and 4, the scope device 102 includes the shaft 104, which is elongated along a longitudinal axis from a proximal end 122 that remains outside the body to the distal end 108 which is inserted to a target site within a living body. The shaft 104 is configured to be inserted, for example, via a natural bodily orifice or a surgical opening through a body lumen to a target site to be treated (e.g., within a kidney or a ureter). The distal end 108 of the embodiment shown in FIG. 1 includes a mounting bracket 138 facilitating a sealed engagement of the distal end 108 with the adapter 106.

In one embodiment, the mounting bracket 138 includes a laterally extending protrusion 140 configured to engage an alignment slot 142 formed on the adapter 106, as will be described in further detail below. The scope device 102 additionally includes an imaging system 118 facilitating navigation of the distal end 108 of the shaft 104 to the target area and for visualization of the target area to direct treatments, etc. as would be understood by those skilled in the art. In one example, the imaging system 118 may include an imager and an LED. In this embodiment, the imager and LED are shown as separate components. It will be understood by those of skill in the art, that the imaging system may include any of a variety of imaging components and, in some embodiments, may include a combined imager and LED.

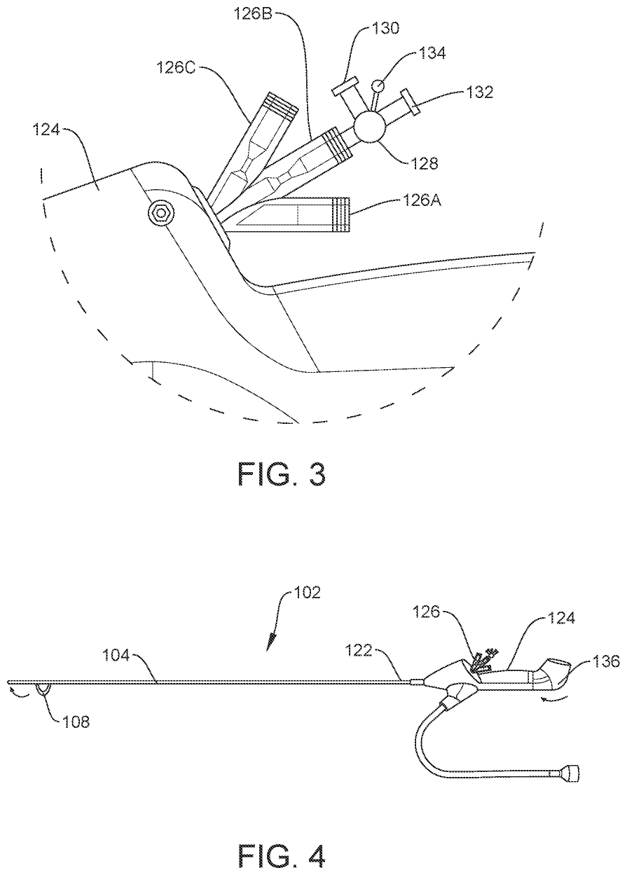

The shaft 104 includes one or more working channels, each of which may be accessed via one or more hubs 126 of a handle member 124 attached to the proximal end 122 of the shaft 104. According to an exemplary embodiment shown in FIG. 3, the shaft 104 includes three working channels and the handle member 124 includes three hubs: a first hub 126A, a second hub 126B, and a third hub 126C; each of which accesses a corresponding one of the working channels. According to an exemplary embodiment, a first working channel used, for example, to provide fluid to the target area, may be accessed via the first hub 126A, while a second working channel used, for example, to apply negative pressure to the distal end of the shaft 104 is accessed via the second hub 126B, and a third channel accommodating, in this embodiment, a laser fiber 120 is accessed via the third hub 126C.

In this embodiment, the second hub 126B (for accessing the second working channel via which a negative pressure is applied) may be fitted with a 3-way stopcock 128 facilitating flushing of the corresponding working channel and the inlet channel 112 of the adapter 106 with which it is in fluid communication and/or aligned. In this embodiment, a first port 130 of the stopcock 128 is connected to a fluid source for flushing the working channel while a second port 132 of the stopcock 128 is connected to a suction source. Thus, the second working channel (and the inlet channel 112 to which it is connected) may be flushed by rotating a stopcock knob 134 to open the first port 130 while closing the second port 132.

The stopcock knob 134 may again be rotated to close the first port 130 so that a suctioning force may be applied through the second port 132. Flushing may be used to clear out clogs, clean the imager lens, and/or increase fluid flow during suctioning. An even further rotation of the stopcock knob 134 may close both ports (i.e., the first port 130 and the second port 132) as would be understood by those skilled in the art.

Although this exemplary embodiment shows and describes the shaft 104 of the scope device 102 as including three working channels, it will be understood by those of skill in the art that this is not a requirement. In some embodiments, a single working channel may serve more than one purpose. For example, the laser fiber 120 may be inserted through the same working channel via which the negative pressure is applied. In this case, the hub 126 corresponding to this working channel may include a Y-connector—a negative pressure source coupled to a first arm of the Y-connector and the laser fiber 120 insertable through a second arm of the Y-connector. If a flushing of the working channel is desired, a 3-way stopcock may be connected to one of the arms of the Y-connector while the other arm of the Y-connector is sealed via a valve such as, for example, a UroLok™ or a Tuohy Borst.

According to an exemplary embodiment, the handle member 124 may further include a deflection knob 136 for deflecting the distal end 108 in, for example, a pigtail configuration, as shown in FIG. 4. In one embodiment, the shaft 104 may be configured for two-way deflections up to approximately 270 degrees in either direction to facilitate navigation to, for example, the poles of the kidney. It will be understood by those of skill in the art that the adapter 106 is attached to the distal end 108 of the shaft 104 such that deflection of the distal end 108 correspondingly deflects the adapter 106 so that it may be pointed in a desired orientation relative to the target area. It will also be understood by those of skill in the art, that the above-described deflections are exemplary only and that, in some embodiments, the scope device 102 may include a rigid shaft rather than the shaft 104 shown and described herein or a shaft that includes rigid and flexible portions in any desired arrangement.

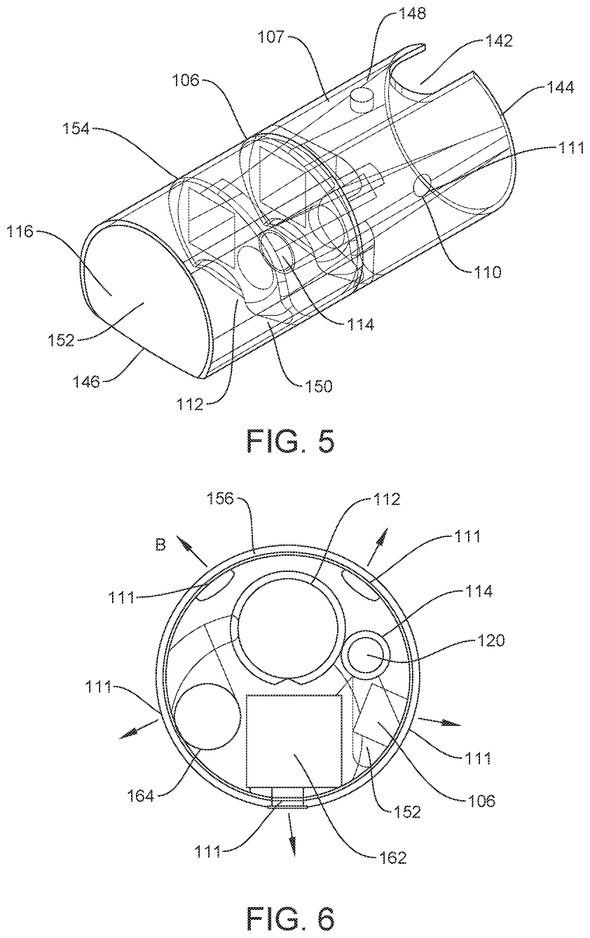

According to an exemplary embodiment, as shown in FIGS. 5-6, the adapter 106 includes a tubular body 107 extending from a proximal end 144 to a distal end 146 and including a lumen 152 therethrough. The adapter 106 includes a proximal portion 148, configured to act as a scope cap over the distal end 108 of the shaft 104 of the scope device 102, and a distal portion 150 including the distal chamber 116. The proximal and distal portions 148, 150 are separated from one another via a partition 154 that extends across the lumen 152. The partition 154 and a wall 156 of the body 107 along the distal portion 150 define the distal chamber 116 within which stone fragments may be received and/or against which stones or stone fragments may be suctioned for lasering, as will be described in further detail below. The wall 156 along the distal portion 150 may include a tapered portion 166, which tapers toward the distal end 146 to facilitate ease of insertion of the adapter 106 through a bodily orifice such as the ureteral orifice.

The proximal portion 148 is configured to be sealingly mounted over the mounting bracket 138 at the distal end 108 of the shaft 104. In one embodiment, the proximal portion 148 may include an alignment slot 142 extending through the wall 156 of the adapter 106, longitudinally from the proximal end 144. The alignment slot 142 is sized and shaped to correspond to the protrusion 140 of the mounting bracket 138 so that, when the protrusion 140 is received and engaged within the alignment slot 142, the adapter 106 is in a desired alignment relative to the shaft 104.

In particular, as will be described in further detail below, when the adapter 106 is in the desired alignment with the shaft 104, the inlet channel 112 will be aligned with the working channel through which the negative pressure (i.e., suction) is applied and the laser channel 114 will be aligned with the working channel through which the laser fiber 120 is inserted. In addition, components of the imaging system 118 such as, for example, an imager and LED, will be properly aligned with corresponding channels/openings of the adapter 106.

According to an exemplary embodiment, the outlet 110 comprises a plurality of apertures 111, with each of the apertures 111 extending through a portion of the wall 156 along the proximal portion 148 of the adapter 106 so that the lumen 152 is open to an exterior of the adapter 106 via the apertures 111. The apertures 111 are positioned between the proximal end 144, which sealingly engages the distal end 108 (e.g., the mounting bracket 138) of the shaft 104, and the partition 154. The adapter 106 is coupled to the shaft 104 so fluid supplied through, for example, the first hub 126A flows through the first working channel to the lumen 152 along the proximal portion 148 of the adapter 106 and out of the apertures 111 of the outlet 110.

In an exemplary embodiment, the apertures 111 are positioned about a perimeter (e.g., circumference) of the proximal portion 148 so that, when fluid is provided through, for example, the first working channel of the shaft 104, the fluid is sprayed circumferentially outward in a pattern B, as shown in FIG. 6, to maintain stone fragments distal of the outlet 110 and to prevent a proximal scattering of the fragments/particles. In one embodiment, the apertures 111 are equidistantly spaced relative to one another about the circumference of the adapter 106. It will also be understood by those of skill in the art, however, that the apertures 111 may extend through the wall 156 along the proximal portion 148 in any of a variety of configurations. It will also be understood by those of skill in the art, that although the outlet 110 is described as comprising a plurality of apertures 111 through the wall 156, the outlet 110 may, in an alternate embodiment, extend to a single aperture extending through the wall 156 in the proximal portion 148 of the adapter 106.

The inlet channel 112 extends from a distal opening 158 in the partition 154 through the lumen 152 longitudinally along the proximal portion 148 to a proximal opening configured to be aligned with and/or engage, for example, the second working channel of the shaft 104 through which negative pressure is applied via the second hub 126B to provide a suction force. The inlet channel 112 is open to the distal chamber 116 so that, when suction is applied therethrough, fluid, stone fragments/particles and/or debris are drawn into and/or against a portion of the distal chamber 116. In one embodiment, the inlet channel 112 may have a substantially funnel-shaped configuration so that stone fragments/particles/debris are funneled into the second working channel.

The laser channel 114 also extends from a distal opening 160 in the partition 154 longitudinally through the lumen 152 along the proximal portion 148 to a proximal end configured to engage, for example, the third working channel of the shaft 104. In this embodiment, the laser fiber 120 may be passed through the third hub 126C and the shaft 104 such that the laser fiber 120 extends distally past the distal opening 160 to be moved into contact with or adjacent to a stone or stone fragment to be lasered. Although the laser channel 114 is described and shown as a laser channel, it will be understood by those of skill in the art that the laser channel 114 may be configured to receive other energy devices and/or retrieval devices.

The adapter 106, in this embodiment, also includes an imager channel 162 and an LED channel 164 extending through the proximal portion 148 for accommodating the corresponding components of the imaging system of the scope device 102. The imager and/or LED channels 162, 164 may be configured as windows extending through the partition 154. It will be understood by those of skill in the art, however, that the imager channel 162 and the LED channel 164 are not required to extend through the partition 154. The adapter 106, however, including the partition 154 and the distal portion 150 is formed of an optically clear (e.g., translucent) material so that imaging via the imaging system is provided therethrough.

It will also be understood by those of skill in the art that while the adapter is shown as including two separate channels 162, 164, the adapter 106 may include any number of channels, openings, or windows for accommodating components of the imaging system 118, depending on the configuration of the imaging system 118. For example, in some embodiments, the imaging system 118 may include a combined imager and LED such that the adapter 106 may include a single window/channel for accommodating the imaging system 118. In addition, although not shown, it will be understood by those of skill in the art that the adapter 106 may be configured to accommodate additional components of a scope device 102 such, as for example, a pressure sensor or other sensor(s). In an exemplary embodiment, a pressure sensor may be used to control a fluid management system of the scope device 102 to manage, for example, inter-renal pressures. The fluid management system may respond to the pressure reading by, for example, increasing or decreasing flow from the outlet 110 and/or increasing or decreasing the negative pressure applied through the inlet channel 112 to adjust the inter-renal pressure. As will be understood by those of skill in the art, complications such as, for example, fever, SIRS, sepsis, postoperative pain, longer hospitalization, and perinephric hematoma, may result from high renal pelvic pressure.

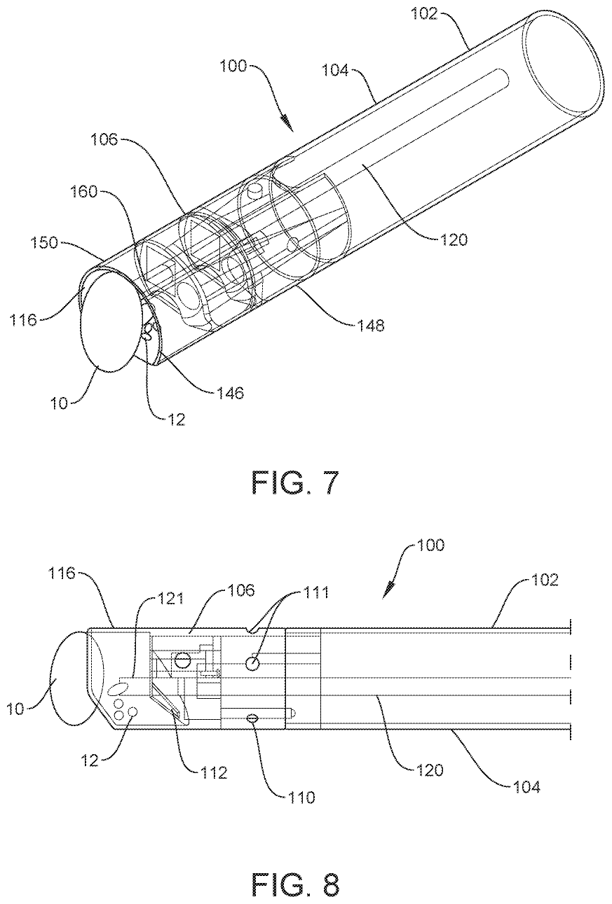

According to an exemplary method for treating, for example, a kidney stone using the system 100, the shaft 104 of the scope device 102, which includes the adapter 106 mounted to the distal end 108 thereof, is inserted through a bodily orifice (e.g., urethral opening) or a surgically created opening to a target area in which a stone or a stone fragment to be treated (hereinafter “a stone 10”) is located. When the distal end of the shaft 104 is positioned as desired relative to a stone 10A, suction force may be applied through the shaft 104 via, for example, the second hub 126B, so that the stone 10 is drawn into and/or against the distal chamber 116, as shown in FIGS. 7-8. When the stone 10 is too large to be drawn proximally into the distal chamber 116, the stone 10 will be pulled by the suction against the distal end 146 of the adapter 106.

In one embodiment, when the stone 10 is held by suction against the distal chamber 116, the shaft 104 may be moved to position the distal end 146 of the adapter 106 so that the stone 10 is in a desired location within the body at which it is desired to dust and/or fragment the stone 10. For example, the stone 10 may be relocated from a lower pole to a mid-pole of the kidney so facilitate the retrieval and removal of fragments from within the kidney.

To treat the stone 10, while the stone 10 is held within or against the distal chamber 116 via the suction force, the laser fiber 120 may be moved distally through the shaft 104 (e.g., via the third hub 126C) so that a distal end 121 of the laser fiber 120 extends distally past the distal opening 160 of the laser channel 114 to contact or come into close proximity with the stone 10. In one embodiment, the laser fiber 120 may be used to fragment the stone 10 so that fragmented portions of the stone 10 are received within the distal chamber 116 and drawn therein via the negative pressure to be removed from the patient's body by withdrawing the scope device 102 from the patient's body. As would be understood by those skilled in the art, this ensures that fragments of the stone 10 may be fragmented until they are small enough to pass through the ureter and/or through an access sheath used to access the target area.

In another embodiment, the laser fiber 120 may be used to dust the stone 10. As the stone 10 is broken into smaller fragments the fragments may be received within the distal chamber 116 so that the stones/stone fragments are allowed to retro pulse or move around within the distal chamber 116 via, for example, a reduction in negative pressure, so that the laser is randomly fired at the stone fragments to break them up to dust size, which may be suctioned through the inlet channel 112 and out of the patient's body.

The negative pressure applied through the inlet channel 112 while the laser fiber 120 is lasering the stone/stone fragments confines the resulting dust particles 12 and heat to within the distal chamber 116. As described above, as the stone 10 is lasered and the negative pressure is applied, fluid is also supplied through the apertures 111 of the outlet 110 to prevent proximal scattering of the dust proximally past the adapter 106. This also generate a fluid circulation (continuous if desired) from the outlet 110 into the body lumen and distally toward the distal end of the adapter 106 through which the fluid enters the distal chamber 116.

The dust/debris and heat are generated within and/or immediately adjacent to the distal chamber 116. Thus, the fluid flowing proximally into the distal chamber 116 absorbs the heat generated through the lasering of the stone 10. This heat is then removed from the area via the heated fluid which is suctioned into the inlet channel 112 through which it passes (along with the dust and stone fragments) out of the patient's body. Since the resulting dust particles 12 is suctioned out of the patient's body, the scope device 102 may remain longer in the target area to treat the next stone/stone fragment, as described above, until all of the stone/stone fragments have been treated and the patient is stone free.

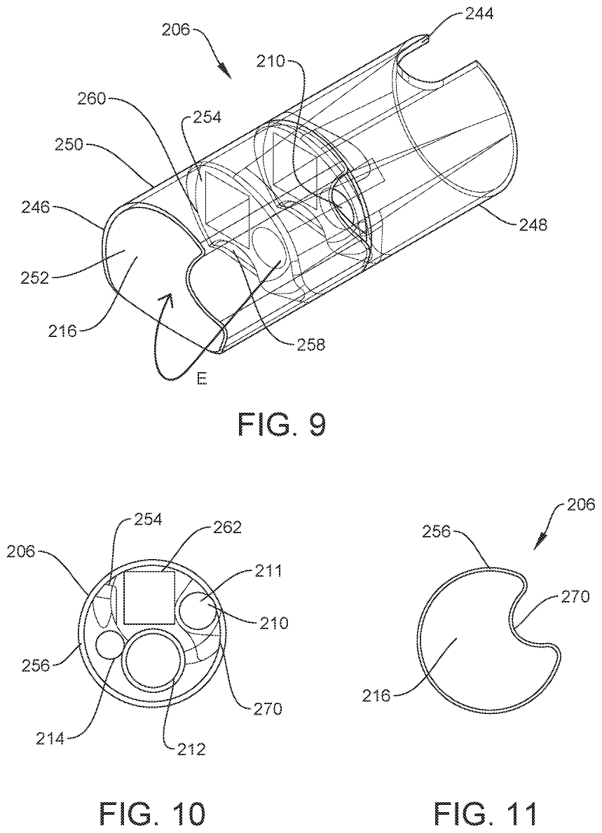

As show in FIGS. 9-11, an adapter 206 according to another exemplary embodiment is a separate item configured to be coupled to a distal end of scope device that may be, other than the adapter 106) similar to the scope device described above in regard to the system 100. Similarly to the adapter 106, the adapter 206 includes an outlet 210 for delivering fluid to a target area and an inlet channel 212 via which a suction force may be applied to provide a continuous fluid flow along a path E, as shown in FIG. 9, distally along an exterior of the adapter 206 to the distal end of the adapter 206 at which the fluid flow is drawn into the adapter 206 via suction applied via the inlet channel 212. The adapter 206 also includes a laser channel 214 through which an energy device such as, for example, a laser fiber may be passed in the same manner described above in regard to the system 100.

Similarly to the adapter 106, the inlet channel 212 and the laser channel 214 are open to and in fluid communication with a distal chamber 216 of the adapter 206 within which stone fragments/particles/debris may be received or suctioned against so that the stone fragments may be treated (e.g., via laser) and suctioned from the target area via the inlet channel 212. The outlet 210 in this embodiment, however, is also configured as a channel 211 extending longitudinally through a portion of the adapter 206, rather than as apertures extending through a circumferential wall thereof as described above with respect to the adapter 106.

In particular, similarly to the adapter 106, the adapter 206 extends longitudinally from a proximal end 244 to a distal end 246 and includes a lumen 252 extending therethrough. The adapter 206 includes a proximal portion 248 and a distal portion 250 separated from one another via a partition 254 extending across the lumen 252. The proximal portion 248 is configured to be mounted over or otherwise coupled to the distal end of the scope device while the distal portion 250 is configured to define a distal chamber 216 therein for receiving stone fragments, particles, and/or debris/dust therein. Similarly to the adapter 106, the inlet channel 212 extends from a distal opening 258 in the partition 254 proximally through the proximal portion 248 to a proximal opening configured so that, when the adapter 206 is mounted on the distal end of the scope or other insertion device to engage a working channel of the scope device, which is configured to receive a negative pressure therethrough.

The laser channel 214 extends from a distal opening 260 in the partition 254 proximally through the proximal portion to a proximal opening configured to engage a working channel of the scope device through which an energy device such as, for example, a laser fiber, may be passed. Thus, similarly to the adapter 106, the inlet channel 212 and the laser channel 214 are in fluid communication with and open to the distal chamber 216. Although the laser channel 214 is described as a laser channel, it will be understood by those of skill in the art that, if so desired, the laser fiber (or other energy device) my share the inlet channel 212 so that the laser channel 214 may be used for the insertion of other tools to the target area. In an alternative embodiment, the laser channel 214 may be entirely eliminated.

The outlet 210 in this embodiment, is also configured as a channel 211 extending from a distal opening 268 in the partition 254 longitudinally through the lumen 252 along the proximal portion 248 to a proximal opening configured to engage a working channel of the scope device through which fluid may be supplied. The outlet 210, however, is not open to and/or in fluid communication with the distal chamber 216 defined within the distal portion 250 of the adapter 206. A wall 256 of the adapter 206 along the distal portion 250 includes a longitudinal indent 270 along an exterior periphery thereof, the longitudinal indent 270 being aligned with the channel 211 of the outlet 210 so that fluid passed through the channel 211 of the outlet 210 is passes distally along the exterior surface of the adapter 206 directly to the target area. This fluid is provided to the target area through the outlet 210 and is then suctioned into the adapter 206 through the distal chamber 216 via the inlet channel 212 along the path B.

It will be understood by those of skill in the art that the adapter 206 may be mounted over, removed from and/or otherwise coupled to and removed from a scope device in a manner to create a scope device that operates in a manner similar to the system 100, as described above, and may be used to treat stones/fragments and/or tissue in a manner substantially similar to the method of use described above for the system 100. It will also be understood by those of skill in the art that, though not specifically described, the adapter 206 may similarly include one or more channels 262 for accommodating components of an imaging system of the scope device in any manner and configuration required to accommodate the components of a scope device for which the adapter 206 is configured.

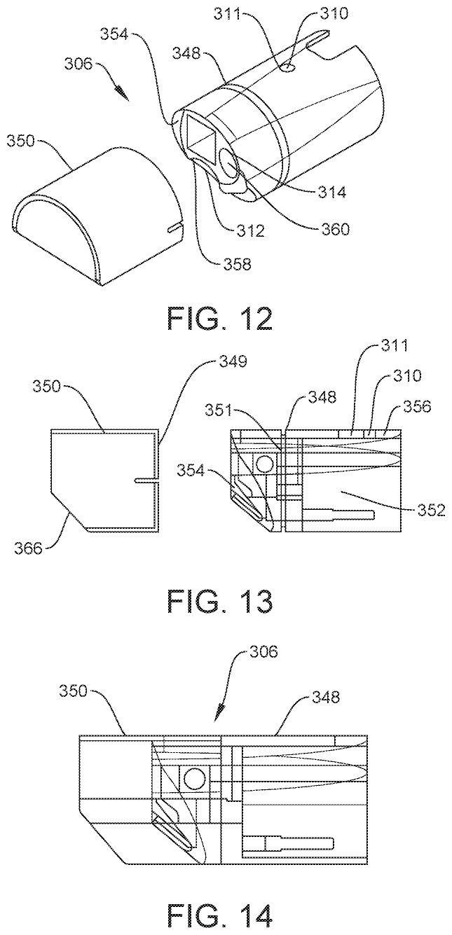

As shown in FIGS. 12-14, an adapter 306 according to another exemplary embodiment of the present disclosure is substantially similar to the adapter 106, as described above, except as delineated below. In contrast the adapter 106, the adapter 306 includes a proximal portion 348 and a distal portion 350 which are configured as two separate components, as shown in FIGS. 12-13, which may be releasably assembled with one another, as shown in FIG. 14) via any of a number of coupling mechanisms including, but not limited to, a friction fit, a snap 349 and groove 351, and threading. The proximal and distal portions 348, 350 may be substantially similar to the proximal and distal portions of the adapter 106. Rather than a partition, however, the proximal portion 348 includes a distal face 354.

The inlet channel 312 extends from a distal opening 358 extending through the distal face 354 longitudinally through a lumen 352 of the proximal portion 348 to a proximal end configured to engage a corresponding working channel (e.g., through which a negative pressure may be applied) of the scope device, as described above. Similarly, the laser channel 314 of this embodiment may be extended from a distal opening 360 extending through the distal face 354 longitudinally through a lumen 352 of the proximal portion 348 to a proximal end configured to engage a corresponding working channel (e.g., through which an energy device may be inserted) of the scope device, as described above. Similarly to the adapter 306, the outlet 310 may be configured as one or more apertures 311 extending through a wall 356 of the proximal portion 348.

The distal portion 350 of this embodiment is substantially similar to the distal portion 150, including features substantially similar thereto such as, for example, a tapered surface 366 at a distal end thereof for ease of insertion through a body orifice. The distal portion 350, however, has a substantially tubular configuration—i.e., a lumen extends therethrough such that a proximal end thereof is open. A distal chamber 316 is not formed thereby until the distal portion 350 is assembled with the proximal portion 348. Once assembled, the distal face 354 of the proximal portion 348 acts similarly to the partition 154 described with respect to the adapter 106, defining a proximal end of the distal chamber 316.

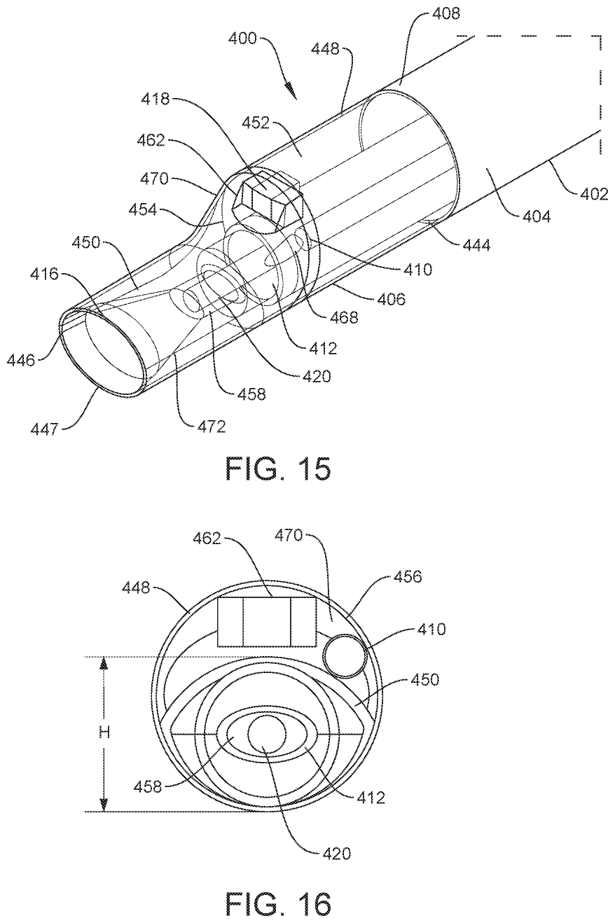

As shown in FIGS. 15-17, a system 400 according to another exemplary embodiment of the present disclosure is, except as distinguished below, substantially similar to the system 100, described above, comprising an adapter 406 coupled to a distal end 408 of a scope device 402 (e.g., ureteroscope) to treat stones or stone fragments within a target area of a patient's body (e.g., kidney, ureter). Similarly to the adapters described above, the adapter 406 include a proximal portion 448 and a distal portion 450, the distal portion 450 defining a distal chamber 416 within which stone fragments/particles/debris may be received or held thereagainst during a lasering or other treatment thereof. The adapter 406 includes an outlet 410 for providing fluid to the target area an inlet channel 412 for applying a suction force to the target area. The outlet 410 and an imaging system 418 of the scope device 402, however, do not extend through and/or are not longitudinally aligned with any portion of the distal portion 450 of the adapter 406.

Similarly to the adapter 106, the adapter 406 extends from a proximal end 444 to a distal end 446 and includes a lumen 452 extending therethrough. The adapter 406 includes the proximal portion 448 and the distal portion 450 which are separated from one another via a partition 454 extending across the lumen 452. Similarly to the adapters described above, a wall 456 of the adapter 406 along the distal portion 450 and the partition 454 define a distal chamber 416 within which stone fragments/particles/debris as well as heated fluids may be contained.

The distal portion 450, however, has a cross-sectional area smaller than that of the proximal portion 448 so that a portion 470 of the partition 454 extends beyond a periphery of the distal portion 450. The outlet 410 extends from a distal opening 468 in this portion 470 of the partition 454 longitudinally through the proximal portion 448 to a proximal end configured to engage a working channel of the scope device 402 through which fluid is configured to be provided to the target area, exterior to the distal portion 450 and/or the distal chamber 416. A reduced cross-section H of the distal portion 450, as shown in FIG. 16, also acts to facilitate ease of insertion of the adapter through a bodily orifice.

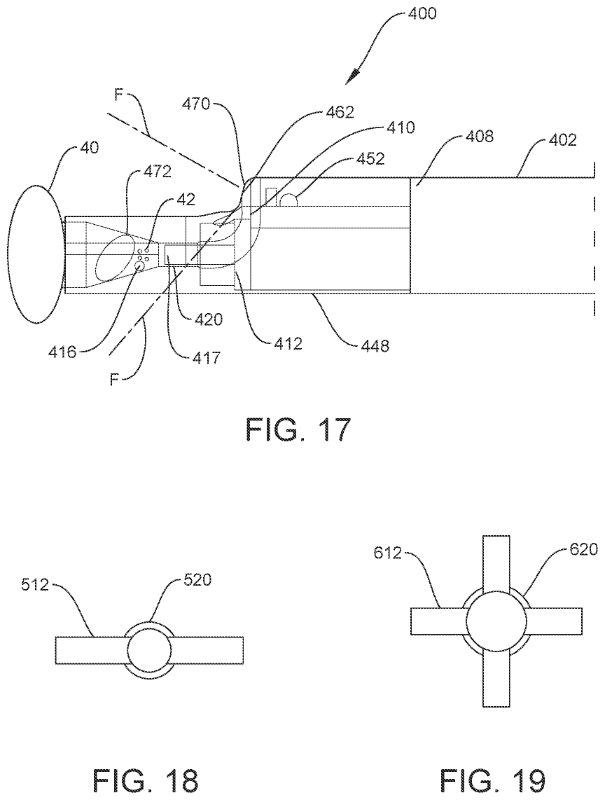

An imager channel or opening 462 also extends through the portion 470 of the partition 454 exterior to the distal portion 450, the imager channel/opening 462 configured to accommodate the imaging system 418 such as, for example, a digital camera or optical lens. The axis of the imager channel or window 426 is exterior to the distal portion 450 and is offset from a longitudinal axis of the proximal portion 448 and/or a shaft 404 of the scope device 402. The imaging system 418, while offset relative to the longitudinal axis of the shaft 404 of the scope device 402, provides a field of view F (shown via dotted lines in FIG. 17) including the distal portion 450 of the adapter 406 and the stones and/or stone fragments being treated. The adapter 406 may be formed of an optically clear (e.g., translucent) material so that the stones/fragments/debris received within the distal chamber 416 are visible therethrough.

Similarly to the adapters described above, the inlet channel 412 extends from a distal opening 458 extending through the partition 454 through the proximal portion 448 to a proximal end configured to engage a working channel of the scope device 402 through which a suction force (i.e., negative pressure) may be applied. The inlet channel 412 is open to and in communication with the distal chamber 416 via the distal opening 458. According to an exemplary embodiment, the inlet channel 412 is configured to also accommodate an energy device such as, for example, a laser fiber 420, therein. In other words, when a laser fiber is received within the inlet channel 412, a negative pressure supplied to the inlet channel 412 would apply suction force about the laser fiber 420.

In an exemplary embodiment, the inlet channel 412 and/or the distal opening 458 thereof may have a non-circular shape cross-section so that, when the laser fiber 420 is received therein, suction force may be applied through the inlet channel 412 to the distal chamber 416 via a space between the laser fiber 420 and the inlet channel 412. In one embodiment, a cross-sectional area of the inlet channel 412 may be substantially elliptical. The cross-sectional area is configured so that, when the laser fiber 420 is received within the inlet channel 412, the laser fiber 420 is substantially centered therein—e.g., substantially aligned with a central axis of the inlet channel 412. According to an exemplary embodiment, a cross-sectional area of the inlet channel 412 may vary along a length thereof. For example, a distal portion of the inlet channel 412 may have an elliptically shaped cross-section while a proximal portion is sized, shaped, and configured to contact, engage or otherwise be aligned with a working channel of the scope device 402.

According to an exemplary embodiment, the distal chamber 416 includes a tapered interior surface 472 which tapers so that an inner diameter of the distal chamber 416 shrinks as a proximal end of the distal chamber 416 is approached—e.g., the diameter increases as the distal opening 458 of the inlet channel 412 is approached. Upon application of suction force through the inlet channel 412, the tapered interior surface 472 funnels stones and/or fragments received therein toward a central axis along which the inlet channel 412 extends so that the stone fragment is substantially aligned with the laser fiber 420.

It will be understood by those of skill in the art that only those fragments, particles, debris, or dust that fit through the clearance between the lase fiber and the inlet channel 412 can pass alongside the laser fiber 420 to be suctioned out of the patient's body. This will prevent any oversized particles or fragments from entering the inlet channel 412 to clog it. It will also be understood by those of skill in the art that any oversized fragments and/or particles at a proximal end of the tapered interior surface 472 may be further fragmented by drawing the laser fiber proximally into the inlet channel 412 and allowing the oversized particle to be drawn into the inlet channel 412 to be further fragmented via lasering.

The system 400 may be used in a manner substantially similar to the system 100. In particular, upon insertion of the shaft 404 through a bodily orifice and to a target area therewithin, a suction force may be applied through the scope device 402 and through the inlet channel 412 so that stones 40 and/or stone fragments may be drawn into the distal chamber 416 and/or drawn against, for example, a distal edge 447 of the distal chamber 416. The laser fiber 420 may be inserted through the inlet channel 412 and into the distal chamber 416 to laser the stone, breaking the stone into smaller fragments, particles, or dust 42.

As the stone/stone fragments are being lasered, fluid is being provided via the outlet 410 and suctioned via the inlet channel 412 to provide a continuous fluid circulation. Thus, those particles that are small enough to clear a space between the exterior of the laser fiber 420 and an interior of the inlet channel 412, along with any heat generated via the lasering, may be removed from the patient's body thereby. As the stone fragments, dust 42 are funneled toward a proximal end 417 of the distal chamber, the laser fiber 420 may be used to continue to further break, fragment and/or pulverize the stone for removal.

As described above with respect to the system 100, in some cases, stone fragments/particles that cannot be suctioned out of the body may be captured and maintained within the distal chamber to be removed from the body upon removal of the scope device 402 from the body. It will also be understood by those of skill in the art, that although not explicitly described with reference to the system 100, stone or stone fragments may be held within or against the distal chamber 416 to be moved or relocated within the body.

Although the inlet channel 412 of the adapter 406 is described and shown as having an elliptical cross-section, it will be understood by those of skill in the art that the inlet channel 412 may have any of a variety of configurations so long as the inlet channel 412 is sized and shaped to center or align a laser fiber, or other energy device received therein, along, for example, the central axis of the inlet channel 412 so that a stone or stone fragment received within the distal chamber 416 may also be aligned therewith for lasering. The inlet channel 412 should also be shaped so that a negative pressure may be applied therethrough to the distal chamber 416, about and/or around the laser fiber 420 received therein.

For example, in another embodiment, as shown in FIG. 18, an inlet channel 512 (e.g., at least a distal portion thereof) has a slotted cross-sectional area configured to center a laser fiber 520 received therein (e.g., substantially along a central axis of the adapter 406 along which the inlet channel 512 extends). Stone particles/debris may be suctioned from the distal chamber via portions of the inlet channel 512 extending from the laser fiber 520. In yet another example, as shown in FIG. 19, a cross-sectional area of an inlet channel 612 (or at least a distal portion thereof) may be substantially cross shaped. The cross-shaped inlet channel 612 may also be configured to center a laser fiber 620 received therewithin, substantially along a central axis along which the inlet channel 612 extends.

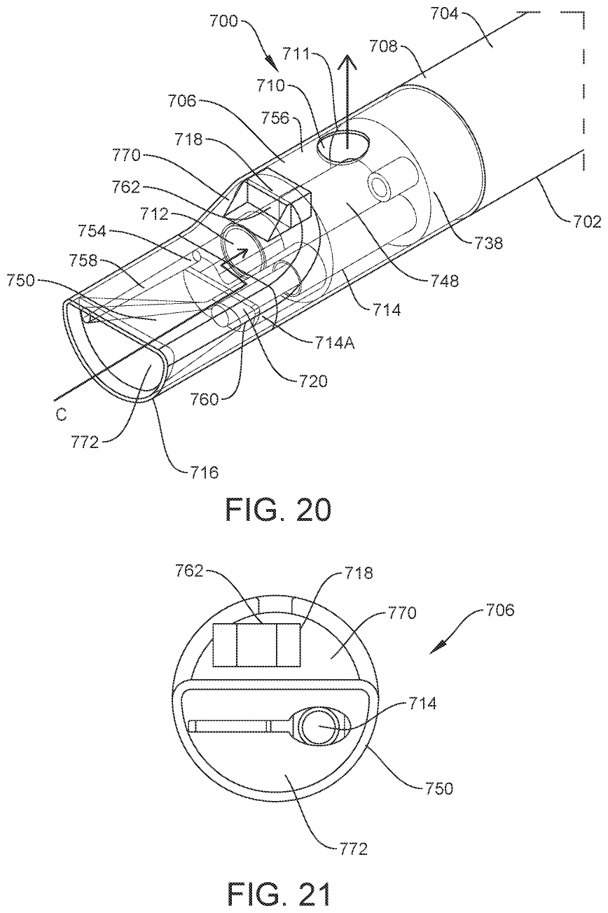

As shown in FIGS. 20-21, a system 700 according to another exemplary embodiment of the present disclosure is substantially similar to the system 400 described above except as described below. The system 700 includes an adapter 706 mounted or otherwise coupled to a distal end 708 of a shaft 704 of a scope device 702 via, for example, a mounting bracket 738 at the distal end 708. The adapter 706 may be substantially similar to the adapter 406, including a distal portion 750 defining a distal chamber 716 within which stones or stone fragments may be drawn into and/or against for lasering, removal or relocation.

Similarly to the adapter 406, a cross-sectional area of the distal portion 750 is smaller than a cross-sectional area of a proximal portion 748 of the adapter 706, the imaging channel 762 for receiving an imager 718 of the scope device 702 extending through a portion 770 of a partition 754 extending laterally beyond the distal portion 750. An outlet 710 in this embodiment, however, may include an aperture 711 extending through a portion of a wall 756 of the proximal portion 748 so that fluid provided therethrough is sprayed radially outward from the adapter 706. Additionally, the adapter 706 includes a separate inlet channel 712 and laser channel 714, as will be described in further detail below.

The laser channel 714 is substantially similar to the inlet channel 412 described above with respect to the adapter 406. Similarly to the adapter 406, the laser channel 714 extends from a distal opening 760 extending through a portion of a partition 754 extending between the distal portion 750 and the proximal portion 748 longitudinally through the proximal portion 748 to a proximal end configured to be aligned with and engage a working channel of the scope device 702 through which the laser fiber 720 is configured to be inserted into the adapter 706. Thus, the laser channel 714 is open to and in communication with the distal chamber 716 via the distal opening 760.

In an exemplary embodiment, at least a distal portion 714A of the laser channel 714 has a non-circular cross-sectional area such that a space exists between the laser fiber 720 received therein and the laser channel 714. In one embodiment, the laser channel 714 has an elliptical cross-section so that a cylindrical laser fiber 720 received therein is centered relative to and/or aligned along a central axis along which the laser channel 714 extends. Also, similarly to the adapter 406, the distal chamber 716 includes a tapered surface 772 configured to funnel stone fragments, particles and debris received therewithin into alignment with the laser fiber 720 to facilitate further lasering of fragments too large to pass through the slotted openings to the inlet channel 712 as described below. In other words, the tapered surface 772 funnels the stone fragments, particles, and debris into alignment with a central axis of the laser channel 714 so that they may be directly exposed to the lasering until they are small enough to pass through the slots into the inlet channel 712.

The inlet channel 712, in this embodiment, extends through the distal portion 750 and the proximal portion 748. A portion 758 of the inlet channel 712 extending through the distal portion 750 has a cross-sectional area sized and shaped to prevent oversized particles from passing therethrough. In one embodiment, the portion 758 of the inlet channel 712 extending through the distal portion has a slotted cross-section while a portion of the inlet channel 712 extending through the proximal portion 748 is sized and shaped to receive and/or engage a working channel of the scope device 702 through which a negative pressure is configured to be applied. Thus, when negative pressure is supplied through the inlet channel 712, fragments or particles that cannot pass through the slotted portion 758 of the inlet channel 712 extending through the distal portion 750 are funneled toward a proximal end of the distal portion 750, into alignment with the laser fiber 720 to facilitate further lasering thereof. As the stone fragments are lasered, particles, debris or dust may pass through the adapter 706 along the path C, shown in FIG. 20.

It will be understood by those of skill in the art that the system 700 may be utilized in a manner substantially similar to the systems 100, 400 described above. As described above with respect to the systems above, fluid provided via the outlet 710 and suction provided via the inlet channel 712 provide a continuous fluid circulation so that stone fragments, particles, debris and/or dust, along with heat generated via the lasering of the stones, are removed from the distal chamber 716 and its immediately surrounding area.

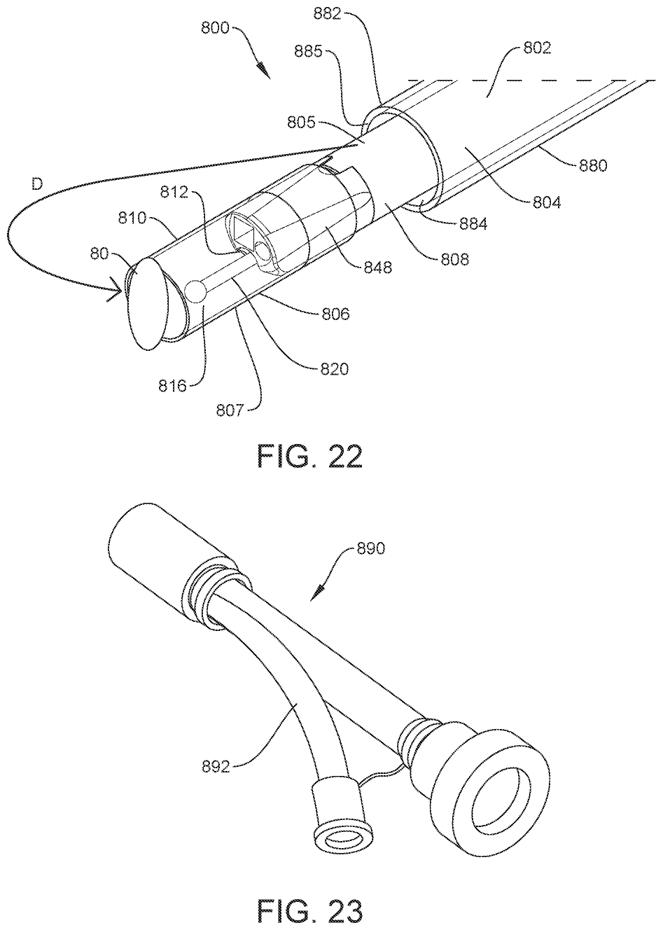

As shown in FIGS. 22-23, a system 800 may be substantially similar to the systems 100, 400 described above, comprising a scope device 802 and an adapter 806 configured to be mounted or otherwise coupled to a distal end 808 of the scope device 802. The system 800, however, may be utilized as a conversion kit for converting a standard scope device 802 including a single working channel, and further includes an outer sheath 880 configured to be slidably placed over a shaft 804 of the scope device 802. The system 800 may be utilized in a manner substantially similar to the systems described above.

As shown in the FIG. 22, the scope device 802 may be a standard scope, in which the distal end 808 of the shaft 804 is preconfigured with a scope cap 848 including an inlet channel 812 aligned with and/or in communication with the working channel of the shaft 804. The adapter 806, in this embodiment, is configured as a tubular body 807 sized, shaped, and configured for mounting over the scope cap 848 via, for example, a friction fit, so that the adapter 806 is removably couplable thereto. Upon mounting of the adapter 806 over the scope cap 848, a distal chamber 816 is formed therein. Although the system 800 is described and shown as a conversion kit for converting the scope device 802, it will be understood by those of skill in the art that the adapter 806 may, similarly to the adapters described above, include a proximal portion configured to be mounted to the distal end 808 of the shaft 804 to act as a scope cap and a distal portion defining a chamber, the proximal portion configured to include channels/openings corresponding to the working channel of the scope device 802 and any components of an imaging system thereof.

The outer sheath 880 extends longitudinally from a proximal end (not shown) to a distal end 882 and includes a lumen 884 extending therethrough. The outer sheath 880 is sized, shaped, and configured to be slidably mountable over a length of the shaft 804. The outer sheath 880 may be placed along the shaft 804 so that the distal end 882 is adjacent or in close proximity to the distal end 808 of the shaft 804. Once the outer sheath 880 has been positioned over the shaft 804 of the scope device 802, as desired, a gateway adapter such as, for example, a Tuohy Borst adapter 890, as shown in FIG. 23, may be tightened against the shaft 804 of the scope device 802 to lock and seal the outer sheath 880 in place.

As will be described in further detail below, a space between an exterior 805 of the shaft 804 and an interior surface 886 of the lumen 884 acts as an outlet to deliver fluid to a target site within a patient's body. The fluid may be provided to the lumen 884 via, for example, a side arm 892 of the Tuohy Borst adapter 890.

Upon assembly of the system 800, as described above, the distal end 808 of the shaft 804, with the adapter 806 coupled thereto, may be inserted through a bodily orifice to a target area (e.g., kidney, ureter) in the patient's body. A negative force may be applied through the working channel of the scope device 802 and the inlet channel 812 of the scope cap 848 so that a stone or stone fragment is suctioned into and/or against the distal chamber 816. A laser fiber 820 may then be inserted through the same working channel and inlet channel 812 to laser the suctioned stone/stone fragment 80. Simultaneously, fluid is provided through the outer sheath 880, via the space between the exterior 805 of the shaft 804 and the interior surface 886 of the lumen 884, to the target area so that fluid circulation (e.g., continuous fluid circulation) along a path D, as shown in FIG. 22, is provided to remove stone fragments, particles, dust/debris, and heat resulting from the lasering of the stone via the working channel.

As described above with respect to the systems 100, 400, 700, this process may be repeated—without removing the scope device 802 from the patient's body—until all of the stones/stone fragments have been treated as desired. As also described above with respect to the systems 100, 400, 700, in some cases, the distal chamber and the suction force applied through the working channel may be used to remove, carry, move and/or relocates stones/stone fragments via the distal chamber 816.

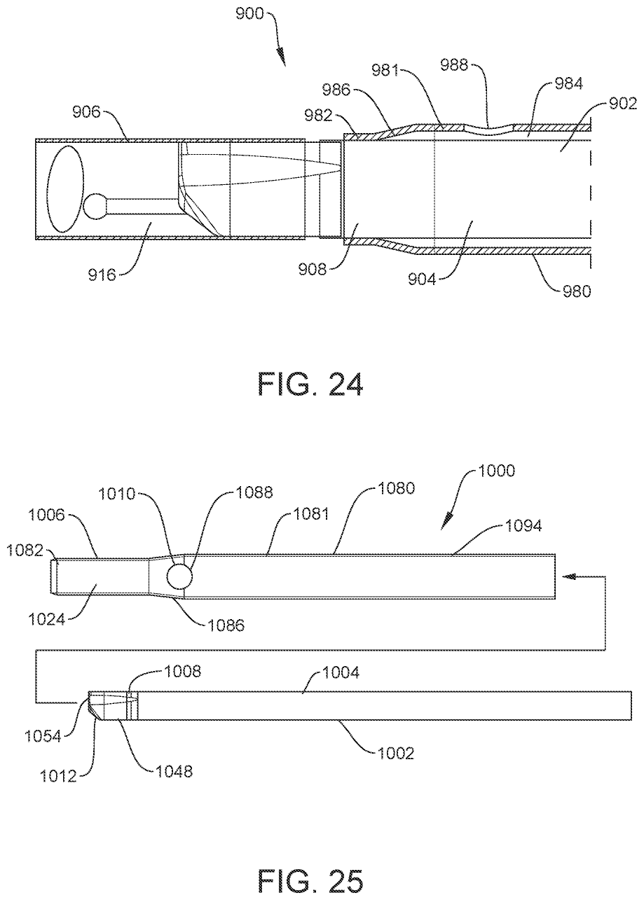

According to another embodiment, as shown in FIG. 24, a system 900 may be substantially similar to the system 800 described above, comprising an adapter 906 and an outer sheath 980 configured to convert a scope device 902 for treating, for example, kidney stones. The adapter 906 is configured to be removably mounted over or otherwise coupled to a distal end 908 of a shaft 904 (e.g., over a scope cap 948) of the scope device 902 while the outer sheath 980 is configured to be slidably mounted over a length of the shaft 904. The adapter 906 and the scope device 902 may be substantially similar to the adapter 806 and the scope device 802 described above with respect to the system 800 (except as pointed out below). In a further embodiment, the adapter 906 may be integrally formed with the outer sheath 980.

In this embodiment, however, the outer sheath 980 includes a taper 986 at a distal end 982 thereof so that the distal end 982 is fitted about the distal end 908 of the shaft 904. The outer sheath 980 includes an outlet opening 988 extending through a wall 981 immediately proximal to the distal end 982 so that fluid passes through a space between an exterior of the shaft 904 and an interior surface of a lumen 984 and out of the outlet opening 988 to a target area of the body into which the distal end 908 (on which an adapter 906 is mounted) has been inserted. The system 900 may be used a manner substantially similar to the system 800 described above, providing continuous fluid circulation from the outlet opening 988, through a distal chamber 916 formed via the adapter 906, and through an inlet channel 912.

According to an alternate embodiment, the distal end 982 may be attached to the shaft 904 to prevent the outer sheath 980 from buckling as the scope device 902 is inserted to the target area via a bodily orifice. In one example, the distal end 982 may be attached to the shaft 904 via an elastic member mounted over the distal end 982.

According to another exemplary embodiment, as shown in FIG. 25, a system 1000 may be utilized as a conversion kit for converting a standard scope device 1002, substantially similarly to the systems 800, 900 described above. Similarly to the systems 800, 900, the scope device 1002 includes a scope cap 1048 pre-mounted over a distal end 1008 of a shaft 1004 so that an inlet channel 1012 of the scope cap 1048 is aligned and in communication with a working channel of the shaft 1004. The system 1000, however, comprises a single outer sheath 1080 configured to be slidably mounted over the scope device 1002, rather than a separate outer sheath and adapter, as described above with respect to the systems 800, 900.

The outer sheath 1080 extends longitudinally from a proximal end (not shown) to a distal end 1082 and includes a lumen 1084 extending therethrough. The outer sheath 1080, in this embodiment, includes a distal portion 1006 configured to extend along and distally of a scope cap 1048 at a distal end 1008 of the scope device 1002 to form a distal chamber 1016. The distal portion 1006 has a smaller diameter than a remaining length 1094 of the outer sheath 1080 so that, when positioned over the scope device 1002 in an operative configuration, the distal portion 1006 is fitted about scope cap 1048 via, for example, a friction fit. As the distal portion 1006 is fitted about the scope cap 1048, the distal chamber 1016 is formed via a portion of the distal portion 1006 extending distally of the scope cap 1048 and a distal face 1054 of the scope cap 1048.

Similarly to the outer sheath 980, the remaining length 1094 may include a tapered distal end 1086 which tapers toward the distal portion 1006. When the distal portion 1006 is fitted about the scope cap 1048 in the operative configuration, the remaining length 1094 of the outer sheath 1080 extends along a length of the shaft 1004. Once the outer sheath 1080 has been positioned over the scope device 1002, as desired, a gateway adapter such as, for example, a Tuohy Borst adapter 890, described above with respect to the system 800, may be tightened over the proximal end of the outer sheath 1080, against the shaft 1004, to lock and seal the outer sheath 1080 in place thereover.

Similarly to the outer sheath 980, the outer sheath 1080 includes an outlet 1010 defined via an outlet opening 1088 extending through a portion of a wall 1081 of the outer sheath 1080, proximal of the distal portion 1006. The outlet opening 1088 extends through the wall 1081 so that fluid may pass through a space between an exterior of the shaft 1004 and an interior surface of the lumen 1084 and out of the outlet opening 1088 to a target area of the body into which the distal end 1008 of the scope device 1002 has been inserted. The system 1000 may be used in a manner substantially similarly to the systems 800, 900, as described above. In an exemplary embodiment, a suction force may be applied through the working channel of the shaft 1004 and through the inlet channel 1012 so that a kidney stone is suctioned into or against the distal chamber 1016. A laser fiber may also be inserted through the inlet channel 1012 to apply laser energy to the stone to fragment and/or pulverize the stone. Fluid is simultaneously provided to the target area so that a continuous fluid circulation is provided from the outlet opening 1088, through the distal chamber 1016, and proximally through the inlet channel 1012 of the scope device 1002 so that stone fragments, particles and/or debris, along with any heated fluids (heated via the lasering of stones or other treatments), may be removed from the target area.

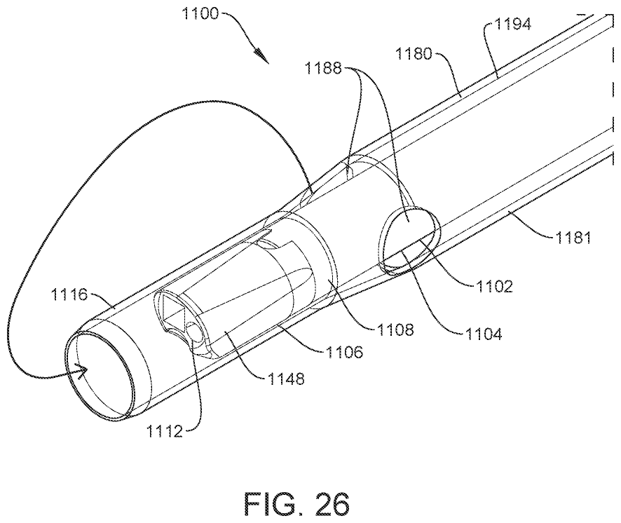

According to another exemplary embodiment, as shown in FIG. 26, a system 1100 may be substantially similar to the system 1000 described above, comprising an outer sheath 1180 configured to convert a scope device 1002 for use in treating kidney stones. The scope device 1102 is substantially similar to the scope device 1002, including a single inlet channel 1112 via which a suction force may be provided to a target area within which a distal end of the scope device 1102 is inserted and via which a laser fiber may be inserted to the target area. The outer sheath 1180 is, substantially similar to the outer sheath 1080, configured to be longitudinally slid over the scope device 1102 so that a distal portion 1106 extends along and distally of a scope cap 1148 at a distal end 1108 of a scope shaft 1104 of the scope device 1102 and a remaining length 1194 extends proximally along a length of the scope shaft 1104. Similarly to the outer sheath 1080, the distal portion 1006 is sized, shaped and configured to have a smaller diameter than the remaining length 1194 so that the distal portion 1006 fits over the scope cap 1148 via, for example, a friction fit, to define a distal chamber 1116 therein.

Rather than a single outlet opening as described with respect to the outer sheath 1080, however, the outer sheath 1180 includes a pair of outlet openings 1188 extending through a wall 1181 of the remaining length 1194, proximate the distal portion 1106. The outlet openings 1188 may additionally have any of a variety of sizes and shapes. In one embodiment, the pair of outlet openings 1188 extend through portions of the remaining length 1194, diametrically opposed to one another. It will be understood by those of skill in the art, however, that the two outlet openings 1188 may have any of a variety of configurations and positions relative to one another so long as the outlet openings 1188 are configured to facilitate a fluid passing from a lumen 1184 thereof, through the outlet openings 1188, to an exterior of the outer sheath 1180—e.g., to a target area within which a distal end of the scope device 1102 is inserted. The system 1100 may be utilized in a manner substantially similar to the systems 800-1000 described above, to provide a continuous fluid circulation from the outlet openings 1188, through the distal chamber 1116, and through an inlet channel 1112 of the scope shaft 1104 to be drawn proximally through the scope device 1102.

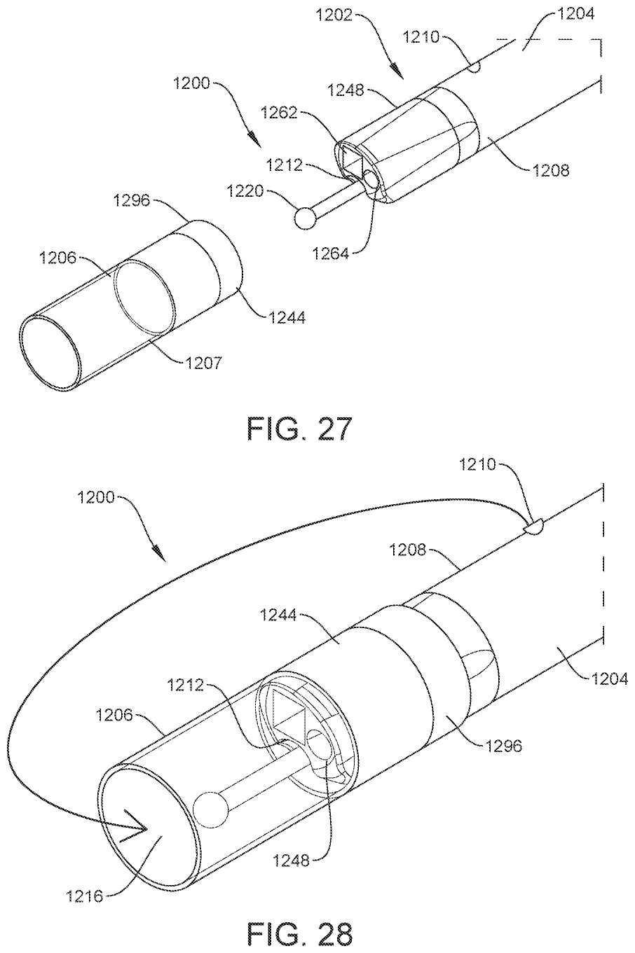

As shown in FIGS. 27-28, a system 1200 according to another exemplary may be substantially similar to the systems 800, 900, as described above, comprising an adapter 1206 configured to convert a scope device 1202 for use in treating, for example, a kidney stone. The system 1200 does not require an outer sheath, however, since the scope device 1202, while substantially similar to the scope devices 802-1102 in other respects, includes two working channels extending through a shaft 1204 thereof—a first working channel for providing suction to a target area within which a distal end of the scope device 1202 is inserted and a second working channel for providing fluid to the target area, as will be described in further detail below.

The scope device 1202 may be substantially similar to the scope devices 802-1102 described above, comprising a shaft 1204 and a scope cap 1248 mounted over a distal end 1208 thereof. The shaft 1204, however, includes two working channels extending longitudinally through the shaft 1204. The first working channel is in communication with an inlet channel 1212 of the scope cap 1248, substantially similarly to the scope devices 802-1102, so that a suction force may be applied therethrough and a laser fiber 1220 may be inserted therein. The scope cap 1248, may be substantially similar to the scope caps described above, including openings 1262, 1264 for accommodating other components of the scope device 1202 such as, for example, an imager and LED of an imaging device.

In an exemplary embodiment, the second working channel, however, is not in communication with any openings/channels in the scope cap 1248. Rather, the shaft 1204 includes an outlet opening 1210 extending through a wall of the shaft 1204 such that the second working channel is open to an exterior of the shaft 1204 via the outlet opening 1210. The outlet opening 1210 may extend through a portion of the shaft 1204 proximate the distal end 1208 so that fluid may be delivered to the target area via the second working channel, the fluid exiting the shaft 1204 via the outlet opening 1210.

The adapter 1206 may be substantially similar to the adapters 806, 906 described above with respect to the systems 800, 900. In particular, the adapter 1206 may have a substantially tubular body 1207 and is sized, shaped and configured to be mounted over the scope cap 1248 so that a portion of the tubular body 1207 extends distally from the scope cap 1248, as shown in FIG. 28, to define a distal chamber 1216. In one exemplary embodiment, the adapter 1206 may be mounted over the scope cap 1248 via a friction fit. In another exemplary embodiment, a proximal end 1244 of the adapter includes an elastic connector 1296 such as, for example, an elastic band that is configured to grip the scope cap 1248 and/or the distal end 1208 of the shaft 1204 when the adapter 1206 is mounted thereover.

It will be understood by those of skill in the art that the system 1200 may be utilized in a manner substantially similar to the systems 800-1100 described above to provide a continuous fluid circulation. In particular, fluid is provided via the outlet opening 1210 extending through the wall of the shaft 1204 and is suctioned through the distal chamber 1216 and the inlet channel 1212 of the shaft 1204 to remove any stone fragments/debris, along with any heat produced via lasering of the stone, from the target area.

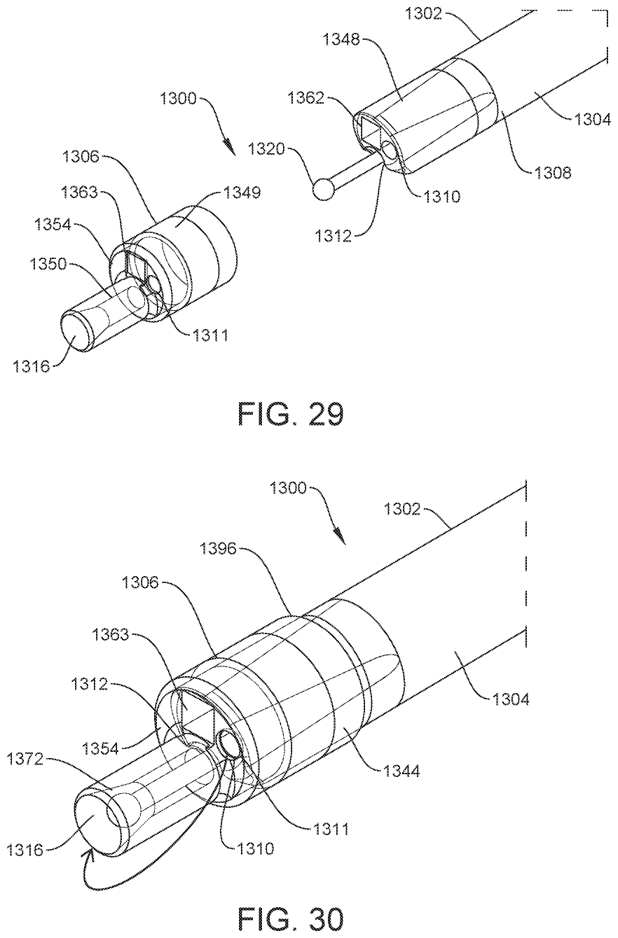

A system 1300, as shown in FIGS. 29-30, is substantially similar to the system 1200 described above, comprising a dual lumen scope device 1302 (i.e., including a first working channel and a second working channel) and an adapter 1306 configured to be mounted over a distal end 1308 thereof. The scope device 1302 is substantially similar to the scope device 1202 described above, including the first working channel extending longitudinally through a shaft 1304 of the scope device 1302 in communication and aligned with an inlet opening 1312 of a scope cap 1348 attached to a distal end of the shaft 1304. In this embodiment, however, rather than an outlet opening extending laterally through a wall of the shaft 1304, the second working channel extending longitudinally through the shaft 1304 is in communication with and aligned with an outlet opening 1310 extending through the scope cap 1348.

In addition, the adapter 1306, includes a proximal portion 1349 configured to be mounted over the distal end 1308 of the scope device 1302 (e.g., over the scope cap 1348) and a distal portion 1350 extending distally from the proximal portion 1349 so that, when the proximal portion 1349 is mounted over the distal end 1308, the distal portion 1350 of the adapter 1306 defines a distal chamber 1316 therein. Similarly to the adapter 1206 described above, the adapter 1306 may include an elastic connector 1396 at a proximal end 1344 of the adapter 1306 for gripping the scope cap 1348 over which the adapter 1306 is mounted.

The distal chamber 1316 is aligned with the inlet opening 1312 so that a target stone may be suctioned against the distal chamber 1316 via a negative force applied through the first working channel and the inlet opening 1312. Similarly to the adapter 406 of the system 400, however, the distal portion 1350 of the adapter 1306 has a smaller cross-sectional area than the proximal portion 1349 such that the outlet opening 1310 and additional openings of the scope cap 1348 such as, for example, an imager opening 1362 for accommodating portions of an imaging system of the scope device 1302, do not extend through and/or are not longitudinally aligned with any portion of the distal portion 1350 of the adapter 1306.

In an exemplary embodiment, a distal face 1354 of the proximal portion 1349 extends beyond a periphery of the distal portion 1350 and includes openings 1363, 1311 which correspond to the imager opening 1362 and the outlet opening 1310, respectively, of the scope cap 1348, when the proximal portion 1349 of the adapter 1306 is mounted over the distal end 1308. In other words, axes along which the imager openings 1362 and the outlet opening 1310 extend through the scope cap 1348 are longitudinally offset from a central axis along which the distal portion 1350 extends from the proximal portion 1349. Thus, fluid is configured to be provided to a target area exterior to the distal portion 1350—i.e., exterior to the distal chamber 1316—and a field of view includes the distal portion 1350. At least the distal portion 1350 of the adapter 1306 may be formed of an optically clear (e.g., translucent) material so that the stones/fragments/debris received within the distal chamber 1316 are visible therethrough.

Similarly to the adapter 406, the distal chamber 1316 of the adapter 1306 may include a tapered interior surface 1372 which tapers so that an inner diameter of the distal chamber 1316 is reduced toward a proximal end of the distal chamber 1316. Thus, as a suction force is applied via the inlet opening 1312, the tapered interior surface 1372 funnels stones, fragments and/or debris toward the central axis along which the inlet opening 1312 extends to align stone fragments with a laser fiber 1320 inserted through the inlet opening 1312.

The system 1300 may be used in a manner substantially similar to the systems described above to provide a continuous circulation for removing fragmented stones/debris and any heat resulting from lasering of a stone from the target area. As shown in FIG. 30, fluid is provided to the target area via the outlet opening 1310 and is suctioned, along with stone fragments/debris and/or any heat resulting from lasering, through the distal chamber 1316 and the inlet opening 1312 to be removed from the body and the target area.

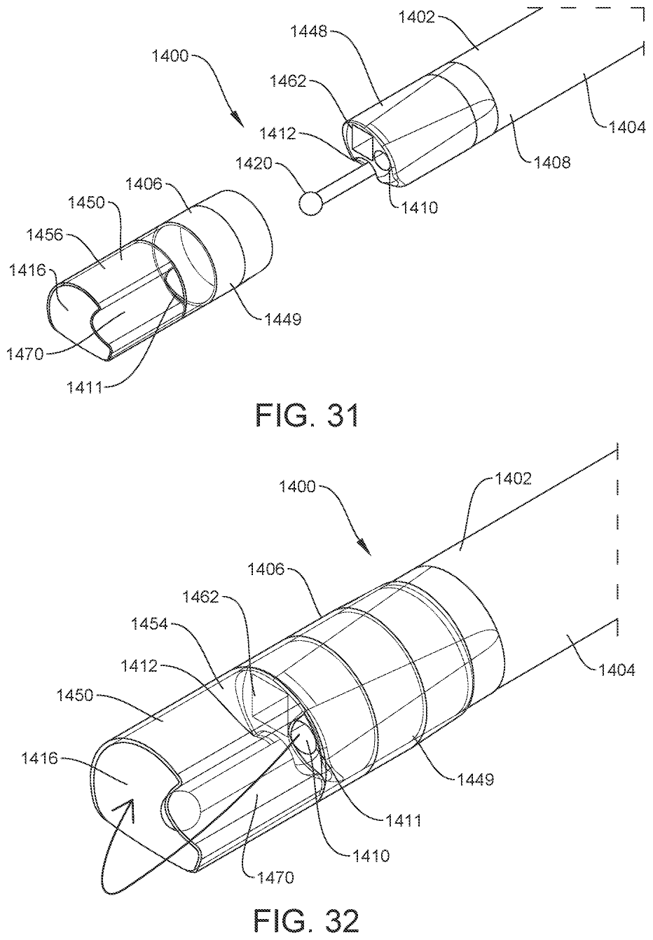

According to another exemplary embodiment, as shown in FIGS. 31-32, a system 1400 according to another exemplary embodiment may be substantially similar to the system 1300 described above, comprising a dual lumen scope device 1402 and an adapter 1406 configured to be placed over a scope cap 1448 that is mounted to a distal end 1408 of a shaft 1404 of the scope device 1402. The scope device 1402 is substantially similar to the scope device 1302, including first and second working channels extending longitudinally through the shaft 1404, the first working channel in communication and aligned with an inlet opening 1412 of the scope cap 1448 and the second working channel in communication and aligned with an outlet opening 1410 of the scope cap 1448. The adapter 1406 may also be substantially similar to the adapter 1306 described above with respect to the system 1300. In particular, the adapter 1406 includes a proximal portion 1449 configured to be mounted over the scope cap 1448 and a distal portion 1450 extending distally therefrom to form a distal chamber 1416 therein, the distal portion 1450 having a smaller cross-sectional area than the proximal portion 1449.

In this embodiment, however, when the adapter 1406 is mounted over the scope cap 1448 in an operative configuration, the distal portion 1450 extends over both the inlet opening 1412 and an imager opening 1462, while the outlet opening 1410 remains offset from the distal portion 1450. In other words, the imager opening 1462 and the inlet opening 1412 are open to and/or in communication with the distal chamber 1416 while the outlet opening 1410 is aligned with a corresponding outlet opening 1411 at a distal end of the proximal portion 1449, which is not in communication and/or aligned with the distal chamber 1416. Thus, fluid is provided to a target area via the outlet opening 1410 exterior to the distal portion 1450 while fluid and/or stone fragments/debris are drawn through the distal chamber 1416 of the distal portion 1450 and proximally through the inlet opening 1412, as shown in FIG. 32.

In an exemplary embodiment, the adapter 1406 may be configured substantially similarly to the adapter 206, as described above. Similarly to the adapter 206, a wall 1456 of the adapter 1406 along the distal portion 1450 includes a longitudinal indent 1470 along an exterior periphery thereof, the longitudinal indent 1470 being aligned with a central axis along which the outlet opening 1411 of the adapter 1406 (and the outlet opening 1410 of the scope device 1402 when the adapter 1406 is mounted thereover in the operative configuration) extends. Thus, as described above, fluid is able to pass through the outlet openings 1410, 1411 distally along the exterior surface (e.g., along the longitudinal indent 1470) directly to the target area. Also similarly to the adapter 206, the wall 1456 along the distal portion 1450 may taper toward a distal end thereof, thereby facilitating ease of insertion of the system 1400 to the target area via, for example, a body orifice.

Similarly to the system 1300, a laser fiber 1420 is described and shown as being passed through the inlet opening 1412 and into the distal chamber 1416 to treat stones and/or stone fragments drawn into and/or thereagainst. As described above with respect to the adapter 206, however, it will be understood by those of skill in the art that the scope device 1402 may include a third working channel, which is configured to be in communication with the distal chamber 1416 in the operative configuration, via which the laser fiber 1420 may be inserted to treat any stones/fragments. The system 1400 may be utilized in a manner substantially similarly to the systems described above, providing a continuous fluid circulation from the outlet opening 1410 and through the distal chamber 1416 and the inlet opening 1412 to draw stone fragments, debris and/or heat away from the target area during treatment (e.g., lasering) of a target stone.

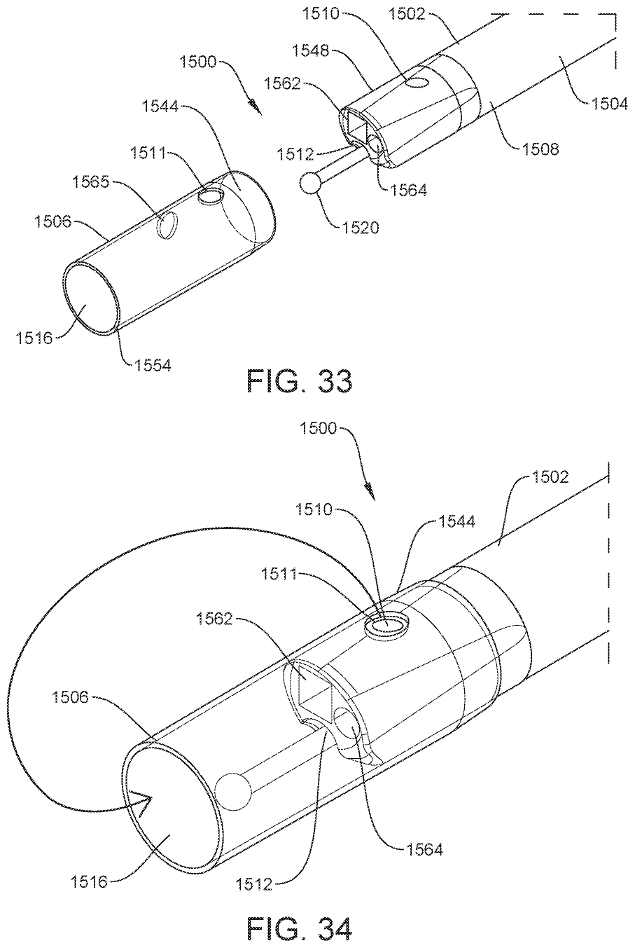

As shown in FIGS. 33-34, a system 1500 according to another exemplary embodiment of the present disclosure may be substantially similar to the system 1200 described above, comprising an adapter 1506 configured to be mounted over a scope cap 1548 at a distal end 1508 of a shaft 1504 of a scope device 1502 to form a distal chamber 1516 therein. The scope device 1502 in this embodiment, however, may include three working channels—a first working channel in communication with and in longitudinal alignment with an inlet opening 1512 and a second working channel in communication with an outlet opening 1510, which extends laterally through a wall of the scope cap 1548.

The scope device 1502 may include a third working channel extending longitudinally through the shaft 1505 in communication with and longitudinally aligned with a corresponding opening 1564 of the scope cap 1548. In this embodiment, a laser fiber 1520 may be inserted through the first working channel and the inlet opening 1512 to the distal chamber 1516, as shown in FIG. 34, or through the third working channel and the corresponding opening 1564 of the adapter 1506 so that an entirety of the inlet opening 1512 is free to suction stone fragments, debris and/or fluid therethrough.

In an exemplary embodiment the adapter 1506 may be substantially similar to the adapter 1206, including a tubularly shaped body 1507, a proximal end 1544 of which is configured to engage the scope cap 1548 via, for example, a friction fit or an elastic connector. The adapter 1506 in this embodiment, further includes an outlet opening 1511 extending through a wall 1554 thereof so that, when the adapter 1506 is mounted over the scope cap 1548 in an operative configuration, the outlet opening 1511 of the adapter 1506 is aligned with the outlet opening 1510 of the scope cap 1548 so that fluid may pass through the second working channel of the scope device 1502 and out of the outlet openings 1510, 1511. The adapter 1506 may additionally include other openings 1565 extending through the wall 1554 and corresponding in size, shape and position to other features of the scope device 1502 such as, for example, a pressure sensor. Alternatively, a pressure sensor may be mounted into the wall 1554 of the adapter 1506 in a desired position therealong.

As described above, the inlet opening 1512 and the corresponding opening 1564 (for accommodating the laser fiber 1520 or another tool) are in communication with and open to the distal chamber 1516 so that stones and/or stone fragments may be treated via, for example, lasering, as described above. Similarly to the systems described above, the system 1500 provides continuous fluid circulation, as shown in FIG. 34, as a target stone is being treated. In particular, fluid is provided to the target area via the outlet openings 1510, 1511 extending laterally through the scope cap 1548 and the adapter 1506. A target stone may be treated via the laser fiber 1520 which may be inserted to the distal chamber 1516 via the inlet opening 1512 or the opening 1564. Resulting stone fragments, debris and/or heat is suctioned from the target area through the distal chamber 1516 and the inlet opening 1512 to be drawn out of the body.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

What is claimed is:

1. A system for treating stones in a hollow organ or body passage, comprising:
an insertion device including a shaft extending longitudinally from a proximal end to a distal end, the shaft being configured for insertion to a target area within a hollow organ or a body passage and including a first working channel and a second working channel extending therethrough; and
an adapter including a proximal portion configured to be coupled to the distal end of the shaft in a desired alignment and a distal portion defining a chamber therein for receiving a target stone to be treated, the proximal portion including an outlet extending through a portion thereof positioned so that, when the adapter is in the desired alignment, a fluid supplied to the first working channel is directed out of the adapter via the outlet to an area surrounding the adapter, the adapter further including an inlet channel extending longitudinally through the proximal portion to a distal opening which is open to the chamber, the inlet channel being configured so that, when negative pressure is applied thereto, the fluid from the area surrounding the adapter is drawn through the distal opening through the inlet channel to the second working channel.

2. The system of claim 1, wherein the adapter includes a substantially tubular body extending from a proximal end to a distal end, a partition extending across a lumen of the substantially tubular body to define the proximal and distal portions.

3. The system of claim 1, wherein the outlet includes an aperture extending through a wall along the proximal portion of the adapter.

4. The system of claim 1, wherein the adapter includes a device channel extending through the proximal portion to a distal opening that is open to the chamber, the device channel configured to receive an energy device therethrough for applying an energy to a target stone received within or against the chamber.

5. The system of claim 1, wherein the proximal portion of the adapter incudes an alignment feature configured to engage a corresponding portion of a mounting bracket attached to the distal end of the shaft so that the adapter is coupled to the shaft in an operative configuration.

6. The system of claim 1, wherein the distal portion of the adapter has a smaller cross-sectional area than the proximal portion, and the outlet is configured as an outlet channel extending longitudinally through the proximal portion to a distal opening extending through a distal face of the proximal portion which extends beyond a periphery of the distal portion.

7. The system of claim 1, wherein the proximal and distal portions of the adapter are configured to be releasably coupled to one another.

8. The system of claim 1, wherein the distal portion of the adapter is formed of a translucent material.

9. The system of claim 1, wherein the proximal portion of the adapter includes a window sized and shaped to receive an imaging system of the insertion device therein so that the chamber is within a field of view of the imaging system.

10. The system of claim 6, wherein the chamber includes a tapered surface tapering toward a proximal end of the chamber to funnel one or more stones received therein toward a central axis along which the outlet channel extends.

11. The system of claim 6, wherein at least a distal portion of the outlet channel has a non-circular cross-sectional area configured to align an energy device received therein toward a central axis along which the inlet channel extends so that a negative force may be applied through the inlet channel, about the energy device.

12. The system of claim 11, wherein the non-circular cross-sectional area is one of an elliptical shape, an elongated slot, or a cross-shape.

13. A system for treating stones in a hollow organ or body passage, comprising:
a ureteroscope including a shaft extending longitudinally from a proximal end to a distal end, the shaft configured to be inserted to a target area within a hollow organ or a body passage and including a first working channel and a second working channel extending therethrough; and
an adapter including a proximal portion configured to be coupled to the distal end of the shaft in a desired alignment and a distal portion defining a chamber therein for receiving a target stone to be treated, the proximal portion including an outlet extending through a portion thereof positioned so that, when the adapter is in the desired alignment, a fluid supplied to the first working channel is directed out of the adapter via the outlet to an area surrounding the adapter, the adapter further including an inlet channel extending longitudinally through the proximal portion to a distal opening which is open to the chamber, the inlet channel being configured so that, when negative pressure is applied thereto, the fluid from the area surrounding the adapter is drawn through the distal opening through the inlet channel to the second working channel.

14. The system of claim 13, further comprising:
a laser fiber configured to be inserted through a third working channel of the ureteroscope to laser stones received within or against the chamber, the third working channel extending through the proximal portion to a distal opening that is open to the chamber.

15. The system of claim 13, further comprising:
an imaging system configured to facilitate navigation of the distal end of the shaft to the target area.

16. A method for treating ureteral or kidney stones comprising:
inserting a scope device, with an adapter coupled to a distal end of a shaft of the scope device, to a target area within a patient's body, the adapter including a proximal portion configured to be coupled to the distal end of the shaft and a distal portion defining a chamber therein, the proximal portion including an outlet in fluid communication with a first working channel of the scope device and an inlet channel extending longitudinally through the proximal portion to a distal opening which is open to the chamber;
applying a negative pressure through a second working channel so that a target stone is suctioned into or against a portion of the chamber;
applying a laser energy to the target stone; and
supplying a fluid to the target area via the outlet to provide a continuous fluid circulation from the outlet, proximally through the chamber and the second working channel to remove any stone particles and heat resulting from a lasering of the target stone from the chamber.

17. The method of claim 16, wherein the laser energy is provided via a laser fiber inserted distally through the shaft to the chamber via one of a device channel extending through the proximal portion of the adapter in communication with the chamber and the inlet channel of the adapter.

18. The method of claim 16, further comprising relocating the target stone, which is suctioned within or against the chamber, to a desired location within the patient's body for lasering.

19. The method of claim 16, wherein supplying the fluid to the target area to provide the continuous fluid circulation includes managing the fluid circulation to control a pressure within the target area resulting from at least one of flushing or relocating at least one of the target stone or stone particles.

20. The method of claim 16, further comprising capturing at least one of stones, stone fragments, or particles too large to pass proximally through an outlet channel within the chamber for removal from the patient's body.
